(19) United States
(12) Patent Application Publication
(10) Pub. No.: US 2006/0004468 A1
(43) Pub. Date: Jan. 5, 2006

(54) Prosthetic pneumatic unit

(76) Inventor: Peter Shu-Chuan Wong, Orangevale, CA (US)

Correspondence Address: RABIN & Berdo, PC, 1101 14TH STREET, NW, SUITE 500, WASHINGTON, DC, 20005, US

(21) Appl. No.: 10/879,277

(22) Filed: Jun. 30, 2004

(51) Int. Cl. A61F 2/64; A61F 2/74

(52) U.S. Cl. 623/44; 188/313

(57) ABSTRACT

A prosthetic pneumatic unit includes a cylinder body, both ends of which are respectively closed by a head part and a bottom part, and a piston is interposed within the cylinder body and defines a first and a second chambers; the cylinder body having a first and a second air passages each having a contraction part at the lower portion thereof; a piston rod connected to the piston; an elastic member received in the second chamber for pushing the piston back to an original position thereof; and a first and a second valve mechanisms respectively received in the first and the second air passages for controlling the respective traveling drags and thus up- and down-traveling velocities of the piston rod. Therefore, the up- and down-traveling velocities of the piston rod thereof can be separately controlled by the adjustments of the first and second valve mechanisms according to users'desires.

### BACKGROUND OF THE INVENTION

1. Field of the Invention

The present invention relates to a buffer air-cylinder apparatus, and more particularly to a prosthetic pneumatic unit, wherein up- and down-traveling velocities of a piston rod thereof can be separately controlled according to users'desires.

2. The Related Art

Buffer air-cylinder apparatuses have been widely applied to control the activities of objects in translation or rotation, for example furniture, lift car-doors, general doors, and prosthetic limbs.

Taiwan Patent Publication Number 85200 discloses a prosthetic pneumatic unit for a prosthetic limb, comprising an air-cylinder, a piston rod, one end of which is connected to a piston of the air-cylinder, and the other end thereof extends outward so as to be connected to a knee joint portion that actuates the piston rod; a separation plate interposed in the interior of the air-cylinder and dividing the air-cylinder chamber into two chambers; an air passage connecting the two chambers, a drag of air passing through which can be controlled by adjusting a valve plug. However, the drags of air passing through the air passage for down and up traveling of the piston rod for the prior art cannot be separately controlled by the adjustment of the valve plug. It means that down- and up-traveling velocities of the piston rod cannot be separately controlled. Therefore, the practicability thereof is largely limited.

Thus, it is desired to have a prosthetic pneumatic unit, which down-and up-traveling velocities of a piston rod thereof can be separately controlled according to users'desires.

### SUMMARY OF THE INVENTION

Therefore, a primary object of the present invention is to provide a prosthetic pneumatic unit, which down- and up-traveling velocities of a piston rod thereof can be separately controlled according to users'desires.

Another object of the present invention is to provide a prosthetic pneumatic unit, which can be applied to other fields.

A further object of the present invention is to provide a prosthetic pneumatic unit having a simple structure and easy to assemble.

To achieve the above objects, a prosthetic pneumatic unit in accordance with the present invention comprises a cylinder body, one end of which is closed by a head part, the other end thereof is closed by a bottom part, and a piston is interposed within the cylinder body and defines a first chamber between the piston and the head part and a second chamber between the piston and the bottom part; the cylinder body having a first air passage and a second air passage each having a contraction part at the lower portion thereof; a piston rod, one end of which is connected with the piston and the other end thereof extends outward from the head part of the cylinder body so as to be connected with an object; an elastic member received in the second chamber for pushing the piston back to an original position thereof; a first valve mechanism received in the first air passage for controlling a traveling drag and thus an up-traveling velocity of the piston rod; and a second valve mechanism received in the second air passage for controlling a traveling drag and thus a down-traveling velocity of the piston rod. Thus, the up- and down-traveling velocities of the piston rod thereof can be separately controlled by the adjustments of the first and second valve mechanisms according to users'desires.

### BRIEF DESCRIPTION OF THE DRAWINGS

The present invention will be apparent to those skilled in the art by reading the following description of preferred embodiments thereof, with reference to the attached drawings, in which:

FIG. 1 is a perspective view of a prosthetic pneumatic unit in accordance with the present invention;

FIG. 2 is an exploded view of the prosthetic pneumatic unit of the present invention;

FIG. 3 is a locally exploded view of the prosthetic pneumatic unit of the present invention; and

FIGS. 4A and 4B respectively show a motion diagram of a first valve mechanism and a second valve mechanism of the present invention when a piston rod thereof travels downward and upward.

### DETAILED DESCRIPTION OF THE PREFERRED EMBODIMENT

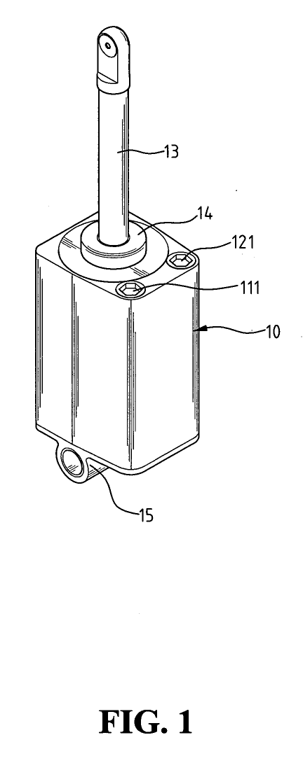

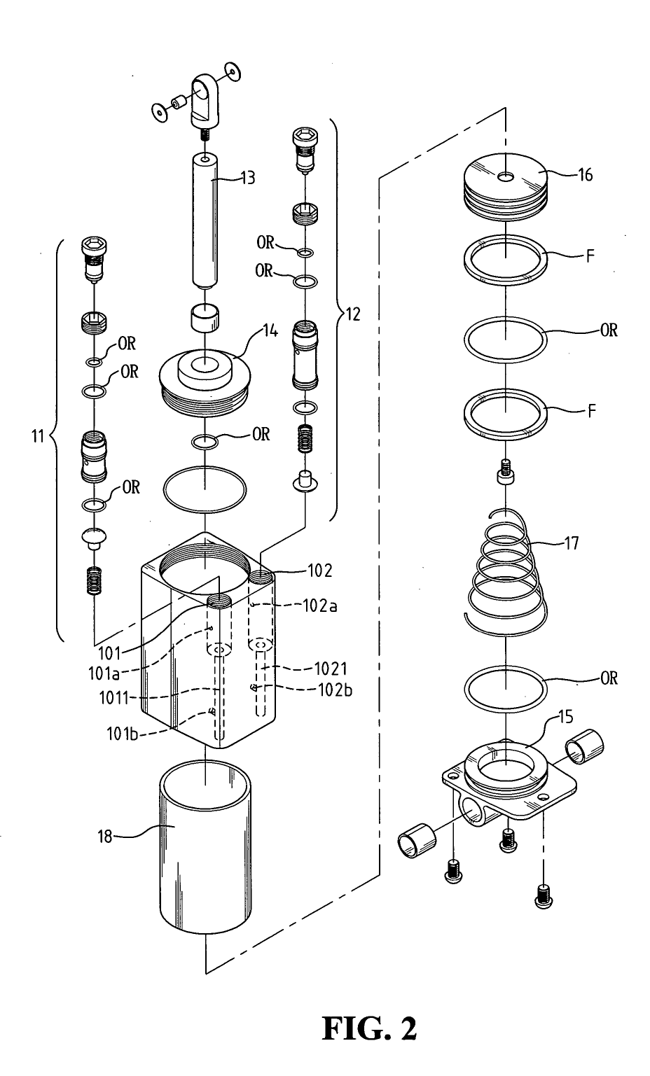

Referring to FIGS. 1 and 2, a prosthetic pneumatic unit in accordance with the present invention comprises a cylinder body 10, one end of which is closed by a head part 14, the other end thereof is closed by a bottom part 15, and a piston 16 is interposed therein and defines a first chamber between the piston 16 and the head part 14 and a second chamber between the piston 16 and the bottom part 15; the cylinder body 10 having a first air passage 101 and a second air passage 102 each having a contraction part 1011, 1021 at the lower portion thereof and holes 101a, 101b, 102a, 102b on side walls of the upper portion and the contraction part of the lower portion thereof interconnecting with the first and second chambers; a piston rod 13, one end of which is connected with the piston 16 and the other end thereof extends outward from the head part 14 of the cylinder body 10 so as to be connected with an object; an elastic member 17 received in the second chamber for pushing the piston 16 back to an original position thereof when an external force applied on the piston rod 13 is released; a first valve mechanism 11 received in the first air passage 101 for controlling a traveling drag and thus an up-traveling velocity of the piston rod 13; and a second valve mechanism 12 received in the second air passage 102 for controlling a traveling drag and thus a down-traveling velocity of the piston rod 13.

Moreover, the present invention further comprises a cylinder bushing 18 received in the cylinder body 10. The piston 16 and the elastic member 17 can move therein.

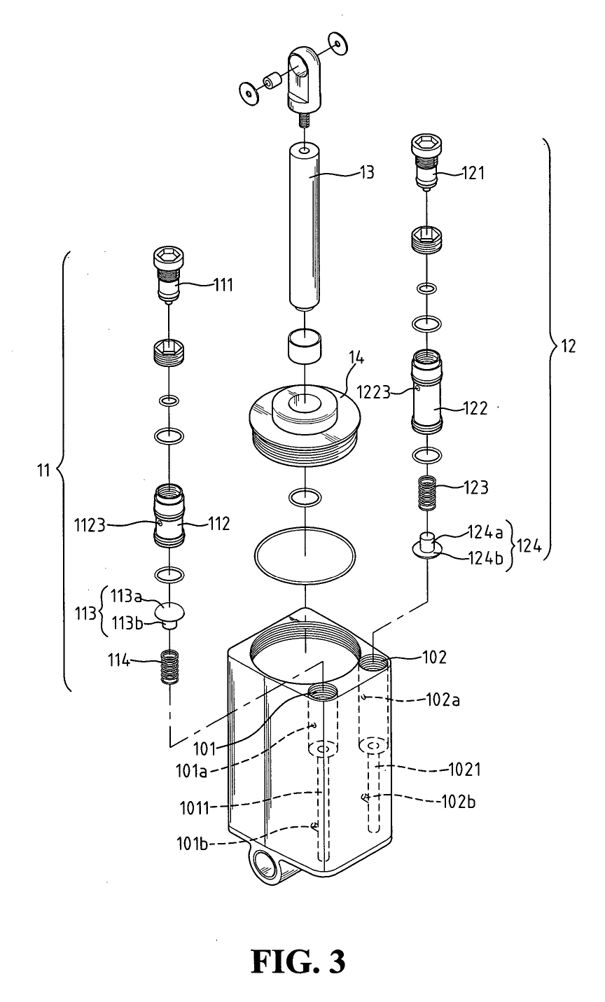

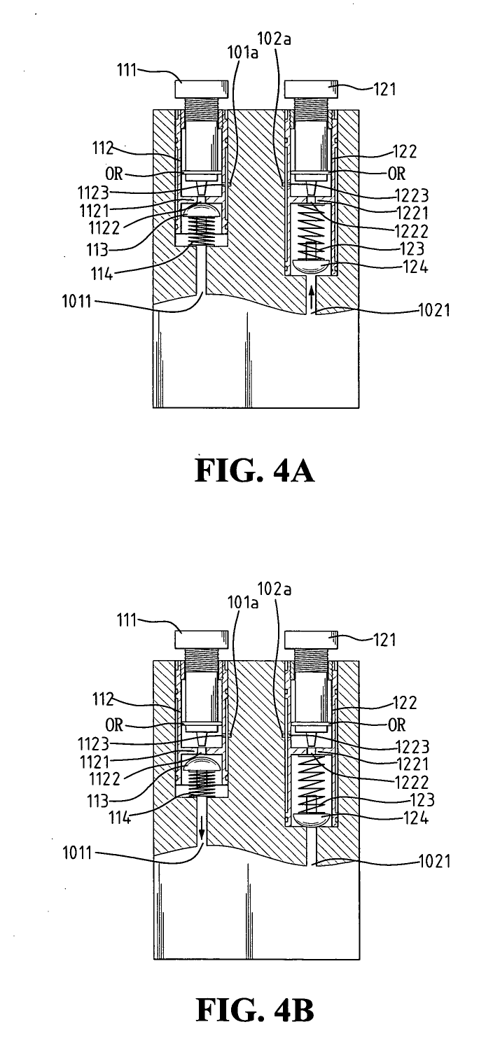

Referring to FIG. 3, the first valve mechanism 11 comprises a first orifice tube 112, a first valve plug 111, a first air door 113, and a first spring 114. The first orifice tube 112 has a first orifice plate 1121 therein and a hole 1123 on a sidewall thereof above the first orifice plate 1121 (see FIG. 4A), which is interconnected with the hole 101a of the first air passage 101 and then the first chamber. One end 113a of the first air door 113 is attached to a first orifice 1122 of the first orifice plate 1121 for controlling the on/off of air passing through there, and the other end 113b thereof is attached to one end of the first spring 114. The other end of the first spring 114 is biased against a top surface of the contraction part 1011 of the first air passage 101. The first valve plug 111 is for example in threaded engagement with the first orifice tube 112. The tip of the first valve plug 111 can be inserted into or removed from the first orifice 1122 of the first orifice plate 1121 by adjusting upward or downward the engagement thereof with the first orifice tube 112 for controlling an flow rate of air passing through the first orifice 1122, a traveling drag and thus an up-traveling velocity of the piston rod 13.

Similarly, the second valve mechanism 12 comprises a second orifice tube 122, a second valve plug 121, a second air door 124, and a second spring 123. The second orifice tube 122 has a second orifice plate 1221 therein and a hole 1223 on a sidewall thereof above the second orifice plate 1221 (see FIG. 4B), which is interconnected with the hole 102a of the second air passage 102 and then the first chamber. One end 124b of the second air door 124 is attached to an top of the contraction part 1021 of the second air passage 102 for controlling the on/off of air passing through there, and the other end 124a thereof is attached to one end of the second spring 123. The other end of the second spring 123 is biased against the second orifice plate 1221. The second valve plug 121 is for example in threaded engagement with the second orifice tube 122. The tip of second valve plug 121 can be inserted into or removed from a second orifice 1222 of the second orifice plate 1221 by adjusting upward or downward the engagement thereof with the second orifice tube 122 for controlling an flow rate of air passing through the second orifice 1222, a traveling drag and thus a down-traveling velocity of the piston rod 13.

Owing that installation positions of the first spring 114 and the first air door 113 of the first valve mechanism 11 are reverse to those of the second spring 123 and the second air door 124 of the second valve mechanism 12, the length of the first orifice tube 112 is preferably shorter than that of the second orifice tube 122 so as to conveniently assemble the springs 114, 123 and the air doors 113, 124 and save material.

In addition, referring to FIG. 2, the present invention further comprises a plurality of O-rings OR or metal rings F, for example Teflon rings, for preventing air leakage.

In operation, referring to FIGS. 4A and 4B, when an external compression force is applied on the piston rod 13, then the piston 16 moves downward. The air inside the second chamber is pressed and passes through in order the hole 102b, the contraction part 1021 of the second air passage 102, the second orifice 1222 of the second orifice tube 122 by the pressed air pushing back the second air door 124, the hole 1223 of the second orifice tube 122 and the hole 102a of the second air passage 102, and finally enters into the first chamber. In contrast to the second air passage 102, owing that the pressed air flows via the hole 101b into the contraction part 1011 of the first air passage 101 and thus pushes the first air door 113 to tightly attach and close the first orifice 1122, the pressed air cannot pass through the first air passage 101.

In reverse, when the external force applied on the piston rod 13 is released, the elastic member 17 provides with a restoring force that makes the piston 16 move backward. The air inside the first chamber is pressed and passes through in order the hole 101a of the first air passage 101, the first air passage 101, the hole 1123 of the first orifice tube 112, the first orifice 1122 of the first orifice tube 112 by the pressed air pushing back the first air door 113, the contraction part 1011 of the first air passage 101 and the hole 101b, and finally back to the second chamber. In contrast to the first air passage 101, owing that the pressed air flows via the hole 102a into the second air passage 102 and then into the second orifice tube 122 and thus pushes the second air door 124 to tightly attach and close the contraction part 1021 of the second air passage 102, the pressed air cannot pass through the second air passage 102.

Also referring to FIGS. 1-3, the openings of the first orifice 1122 and the second orifice 1222 can be controlled by adjusting upward or downward the engagement of the valve plugs 111, 121 with the corresponding orifice tube 112, 122 thereof. Therefore, the drags of air passing through the first and second air passages 101, 102 and thus the up- and down-traveling velocities of the piston rod 13 can be suitably controlled according to users'desires.

Seen from the aforesaid descriptions, the present invention has the advantages of a structure simple and easy to assemble, a wide applications and being able to separately adjust the up- and down-traveling velocities of the piston rod thereof.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A prosthetic pneumatic unit comprising:
a cylinder body, one end of which is closed by a head part, the other end thereof is closed by a bottom part, and a piston is interposed within the cylinder body and defines a first chamber between the piston and the head part and a second chamber between the piston and the bottom part; the cylinder body having a first air passage and a second air passage each having a contraction part at the lower portion thereof and holes on side walls of the upper portion and the contraction part of the lower portion thereof interconnecting with the first and second chambers;
a piston rod, one end of which is connected with the piston and the other end thereof extends outward from the head part of the cylinder body so as to be connected with an object;
an elastic member received in the second chamber for pushing the piston back to an original position thereof;
a first valve mechanism received in the first air passage for controlling a traveling drag and thus an up-traveling velocity of the piston rod; and
a second valve mechanism received in the second air passage for controlling a traveling and thus a down-traveling velocity of the piston rod.

2. The prosthetic pneumatic unit as claimed in claim 1 further comprising a cylinder bushing provided for the piston and the elastic member moving therein.

3. The prosthetic pneumatic unit as claimed in claim 1, wherein the first valve mechanism comprises:
a first orifice tube having a first orifice plate therein and a hole on a sidewall thereof above the first orifice plate, which is interconnected with the hole of the first air passage and then the first chamber;
a first valve plug being in threaded engagement with the first orifice tube, which tip is inserted into or removed from an orifice of the first orifice plate by adjusting upward or downward the engagement thereof with the first orifice tube for controlling an flow rate of air passing through the first orifice, a traveling drag and thus an up-traveling velocity of the piston rod;
a first air door, one end of which is attached to the first orifice of the first orifice plate for controlling the on/off of air passing through there;
a first spring, one end of which is attached to the other end of the first air door, and the other end thereof is biased against the top surface of the contraction part of the first air passage.

4. The prosthetic pneumatic unit as claimed in claim 3, wherein the second valve mechanism comprises:
a second orifice tube having a second orifice plate therein and a hole on a sidewall thereof above the second orifice plate, which is interconnected with the hole of the second air passage and then the first chamber;
a second valve plug being in threaded engagement with the second orifice tube, which tip is inserted into or removed from a second orifice of the second orifice plate by adjusting upward or downward the engagement thereof with the second orifice tube for controlling an flow rate of air passing through the second orifice, a traveling drag and thus a down-traveling velocity of the piston rod;
a second air door, one end of which is attached to the top of the contraction part of the second air passage for controlling the on/off of air passing through there;
a second spring, one end of which is attached to the other end of the second air door, and the other end thereof is biased against the second orifice plate.

5. The prosthetic pneumatic unit as claimed in claim 4, wherein the length of the first orifice tube is shorter than that of the second orifice tube.
